(19) United States
(12) Patent Application Publication
(10) Pub. No.: US 2018/0169289 A1
(43) Pub. Date: Jun. 21, 2018

(54) ATOMIZING ASSEMBLY AND ATOMIZER HAVING THE SAME

(71) Applicant: Taiwan Puritic Corp., Hsinchu County (TW)

(72) Inventor: Chun-Tai CHIANG, New Taipei City (TW)

(21) Appl. No.: 15/900,098

(22) Filed: Feb. 20, 2018

Related U.S. Application Data
(63) Continuation-in-part of application No. 14/882,103, filed on Oct. 13, 2015.

(30) Foreign Application Priority Data
Jul. 29, 2015 (TW) 104212164

(51) Int. Cl. A61L 9/14; B05B 17/00

(52) U.S. Cl. CPC A61L 9/14; B05B 17/0653; B05B 17/0638

(57) ABSTRACT

The present invention relates to an atomizing assembly and an atomizer having the same. The atomizing assembly comprises a containing part and an atomizing part. The containing part includes a containing body defining a containing space, and the containing body has a recess with a first pattern, which is one-piece integrally-formed on the recess. The atomizing part includes a nozzle plate, the elastic element and an atomizing electric-connection portion electrically connected to the nozzle plate. The elastic element inseparable encapsulates part of the nozzle plate. The elastic element has a second pattern corresponding to the first pattern, and the second pattern is closely press-fitted to the first pattern to make the nozzle plate detachably installed in the recess of the containing body. The present invention is easy to assemble/disassemble, and the parts thereof can be closely press-fitted to each other without using any fixing element.

### BACKGROUND OF THE INVENTION

### 1. Field of the Invention

The present invention relates to an atomizing assembly and an atomizer having the same, particularly to an atomizing assembly whose containing part and atomizing part can be press-fitted to or separated from each other without using additional elements, and an atomizer having the same.

### 2. Description of the Prior Art

Generally to speak, an oscillation type atomizer uses a nozzle plate and an oscillation plate to convert a liquid into tiny liquid droplets and spray them out. In a conventional oscillation type atomizer, the atomizing assembly and the atomizer body are fabricated integrally. Thus, the conventional oscillation type atomizer is likely to have malfunctions or current leakage caused by liquid infiltration while it is resupplied with liquid. Further, the user may experience inconvenience in replacing related parts.

Some conventional designs were proposed to solve the abovementioned problems, wherein the atomizing assembly and the atomizer body are fabricated into separate structures. In these improved designs, the joint regions of the atomizing assembly and the atomizer body are normally planar structures. While the atomizer is operating, oscillation converts the liquid into mist. However, oscillation force may also cause the liquid to overflow from the gap between the planer joint regions, which would waste the liquid and may damage the device.

Some conventional designs, such as a kind of desktop atomizer, were proposed to solve the abovementioned problems by using an additional fixing elements. Please refer to FIG. 1 and FIG. 2, which shows an exploded perspective view of the internal construction of the liquid atomizer assembly in U.S. Pat. No. 6,450,419. The piezoelectric actuator and orifice plate pump assembly 42 is positioned on the shelf 40, the retainer 50 is snapped onto the upper cylindrical support wall 44 so that the retainer finger 54 press down on the piezoelectric actuator and orifice plate pump assembly 42 near its outer edge to hold it securely on the shelf. However, the problem of liquid leakage may not be totally overcame.

### SUMMARY OF THE INVENTION

In order to overcome the drawbacks of the conventional technology, the present invention proposes an atomizing assembly and an atomizer having the same, wherein the containing part and the atomizing part of the atomizing assembly have corresponding press-fit structures, whereby the containing part and the atomizing part are easy to assemble and disassemble, and whereby the containing part and the atomizing part can be closely press-fitted to each other without using additional fixing elements, and whereby are avoided the problems of liquid waste and device damage caused by liquid leakage occurring in the conventional atomizing assembly.

One objective of the present invention is to provide an atomizing assembly, which comprises a containing part including a containing body defining a containing space for containing a liquid to be atomized. The atomizing assembly also comprises a nozzle plate, an atomizing electric-connection portion and an elastic element. The nozzle plate comprises a fine spray orifice portion disposed thereon to converts the liquid into mist by oscillation. The atomizing electric-connection portion electrically connected with the nozzle plate. The elastic element inseparable encapsulates the nozzle plate and expose the fine spray orifice portion by two openings on the opposite side. Wherein the elastic element has a second pattern corresponding to the first pattern on one side. The second pattern of the elastic element is closely press-fitted to the first pattern of the containing body to make the nozzle plate detachably installed in the recess of said containing body.

In one embodiment, the first pattern is a convex pattern, and the second pattern is a concave pattern; the convex pattern matches the concave pattern.

In one embodiment, the first pattern is a concave pattern, and the second pattern is a convex pattern; the concave pattern matches the convex pattern.

In one embodiment, the first pattern and the second pattern are annular patterns.

In one embodiment, the atomizing assembly further comprises a fixing cover, whose shape is corresponding to the nozzle plate and the atomizing electric-connection portion, and which is used to cover the nozzle plate and the atomizing electric-connection portion and fix them to the containing body.

In one embodiment, the material of the elastic element comprises rubber.

Another objective of the present invention is to provide an atomizer, which comprises the abovementioned atomizing assembly and a base seat, wherein the base seat includes a base electric-connection portion that is fabricated into a structure able to be electrically connected with the atomizing electric-connection portion.

Below, embodiments are described in detail in cooperation with the attached drawings to make easily understood the objectives, technical contents, characteristics and accomplishments of the present invention.

### BRIEF DESCRIPTION OF THE DRAWINGS

FIG. 1 is an exploded perspective view shows a desktop liquid atomizer assembly in prior art.

FIG. 2 is a cross-section view shows part of the desktop liquid atomizer assembly in prior art.

FIG. 3 is a diagram schematically showing an atomizing assembly according to one embodiment of the present invention.

FIG. 4 is a cross-sectional view of one embodiment of present invention along line A-A′ in FIG. 3.

FIGS. 5A and 5B are schematic diagram schematically showing an atomizing assembly according to one embodiment of the present invention.

FIGS. 5C and 5D are schematic diagram schematically showing an atomizing assembly according to another embodiment of the present invention.

FIG. 6 is a diagram schematically showing an atomizer according to one embodiment of the present invention.

FIG. 7 is a diagram schematically showing a base seat of an atomizer according to one embodiment of the present invention.

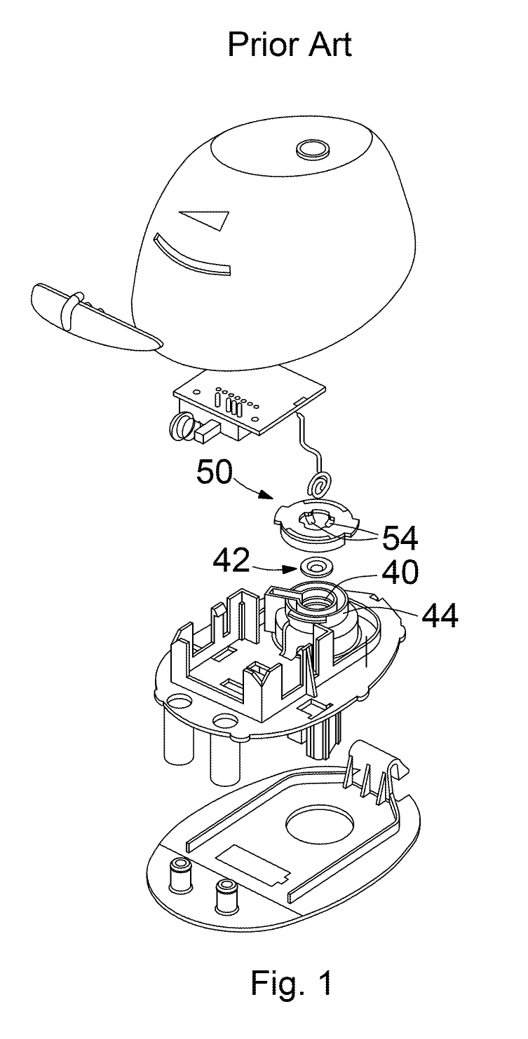

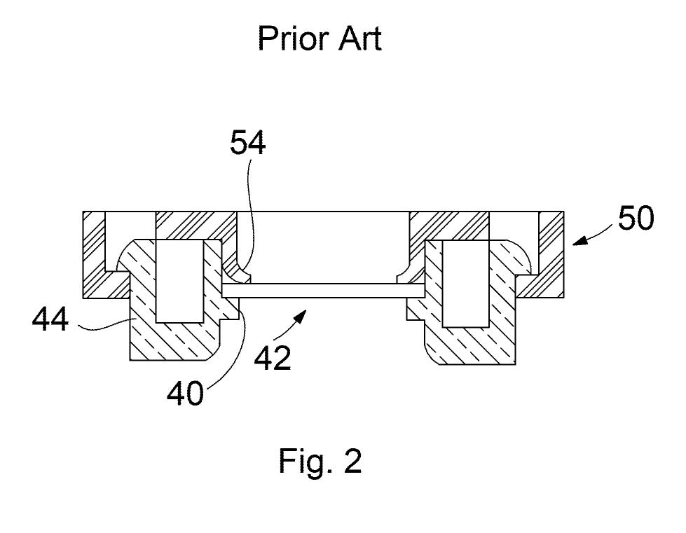

### DESCRIPTION OF THE PREFERRED EMBODIMENT

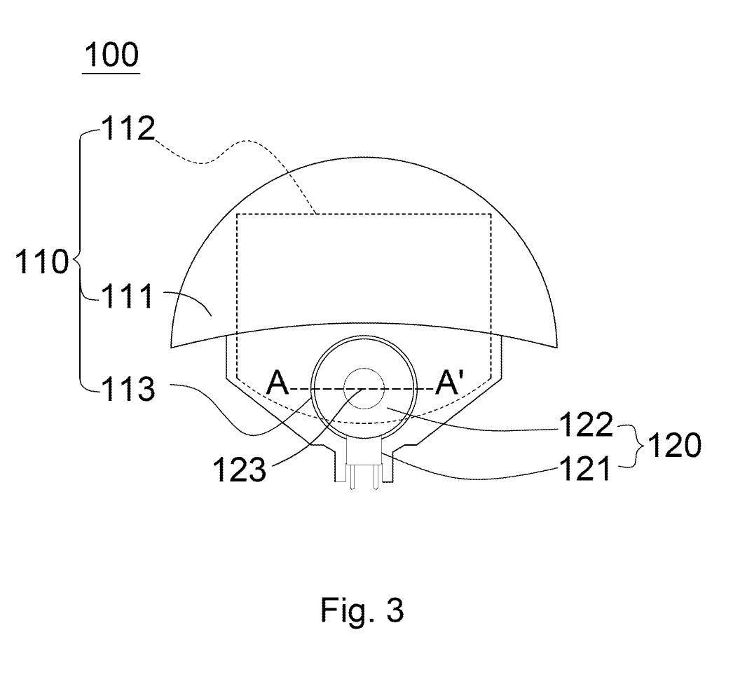

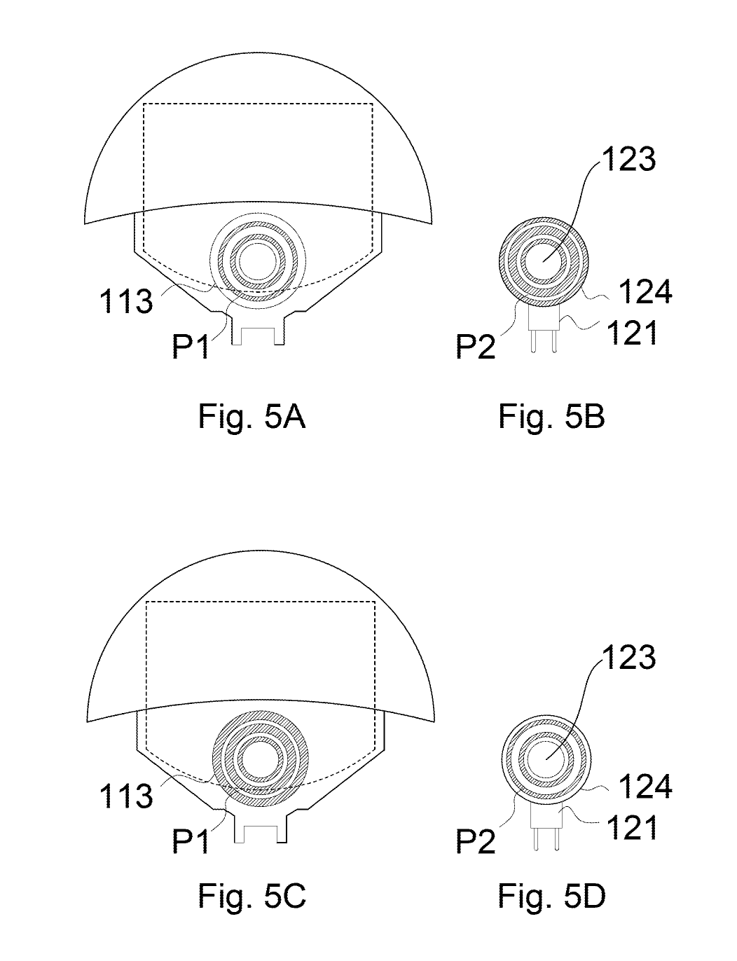

Refer to FIG. 3. The atomizing assembly 100 of the present invention comprises a containing part 110 and an atomizing part 120. The containing part 110 includes a containing body 111 defining a containing space 112 for containing a liquid (not shown in the drawing) to be atomized. The containing body 111 has a recess 113 with a first pattern P1 (as shown in FIG. 5A and FIG. 5B), and the first pattern P1 is one-piece integrally-formed on the recess 113, which will be described in detail hereinafter. The atomizing part 120 of the atomizing assembly 100 includes a nozzle plate 122 and an atomizing electric-connection portion 121 electrically connected with the nozzle plate 122. In one embodiment, the nozzle plate 122 is disposed corresponding to the recess 113 of the containing body 111. The nozzle plate 122 comprising a fine spray orifice portion 123 disposed thereon to converts the liquid into mist by oscillation.

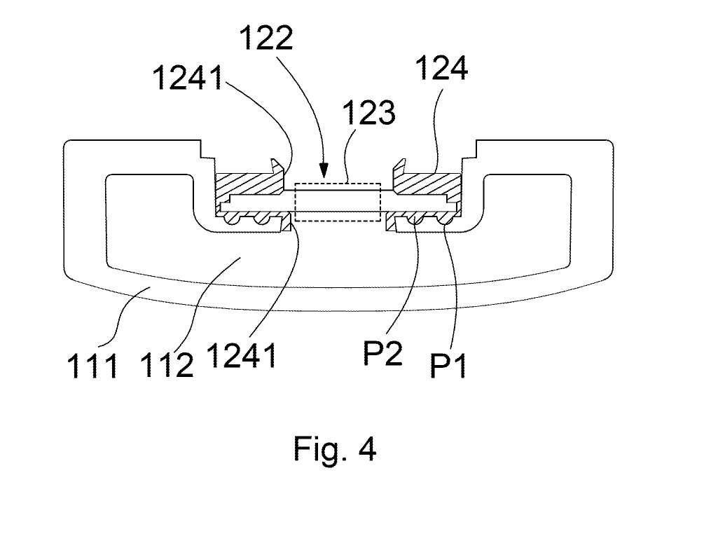

Please refer to FIG. 4. The atomizing part also comprises an elastic element 124 having two openings 1241 on the opposite side. The elastic element 124 inseparable encapsulates the nozzle plate 122 and expose the fine spray orifice portion 123 by two openings 1241. The elastic element 124 comprises a second pattern P2 corresponding to the first pattern P1 on one side, and the second pattern P2 of the elastic element 124 is closely press-fitted to the first pattern P1 of the containing body 111 to make the nozzle plate 122 detachably installed in said recess of said containing body 111.

In one embodiment, the material of said elastic element comprises rubber or other flexible material. It is important that, compare to other material, the material with flexibility of the elastic element may encapsulate the nozzle plate more closely. As such, the close fit between the elastic element and the nozzle plate may prevent the liquid leakage while the atomizer is operating. Furthermore, the second pattern of elastic element may also close fit with the first pattern of containing body, not only by the corresponding shapes of the patterns, but also by the flexible material of the elastic element.

Refer to FIGS. 5A to 5D. The elastic element 124 has a second pattern P2 corresponding to the first pattern P1 of the recess 113. The nozzle plate 122 is detachably installed in the recess 113 of the containing body 111 via press-fitting the second pattern P2 of the elastic element 124 to the first pattern P1 of the recess 113 without using additional elements. In one embodiment, the first pattern P1 is a convex pattern, and the second pattern P2 is a concave pattern; the convex pattern is corresponding to the concave pattern and can be press-fitted to the concave pattern. In another embodiment, the first pattern P1 is a concave pattern, and the second pattern P2 is a convex pattern; the convex pattern is corresponding to the concave pattern and can be press-fitted to the concave pattern. It should be noted: the first pattern P1 and the second pattern P2, which are drawn to have annular shapes in the drawings, are only for exemplification. As long as the first pattern P1 and the second pattern P2 are corresponding to each other and can be press-fitted to each other, the present invention does not limit that the first pattern and the second pattern must have a specified shape.

In one embodiment, the atomizing assembly of the present invention further comprises a fixing cover 130, as shown in FIG. 5. The fixing cover 130 is corresponding to the atomizing part 120. In other words, the shape of the fixing cover 130 is corresponding to the shapes of the nozzle plate 122 and the atomizing electric-connection portion 121, and the fixing cover 130 is used to cover the nozzle plate 122 and the atomizing electric-connection portion 121 and fix them to the containing part 110. In one embodiment, an adhesive agent or fixing elements are applied between the fixing cover 130 and the atomizing part 120 to enhance the connection and leakage resistance thereof.

It is noted that the fixing cover and the adhesive agent are used to enhance the connection between the nozzle plate and containing part for a better fixed effect, however, the fixing cover and the adhesive agent are optional elements depending on the situation. Indeed, the containing part and the atomizing part of the present invention can be closely press-fitted to each other by the first pattern and second pattern, which are one-piece integrally-formed thereon, respectively, without using additional fixing elements.

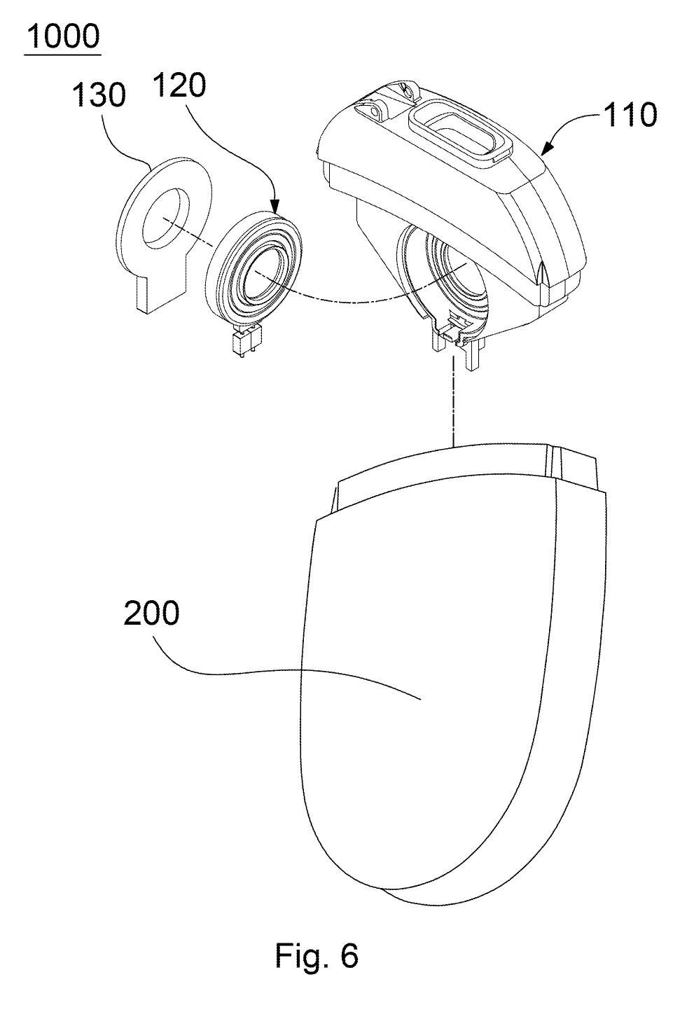

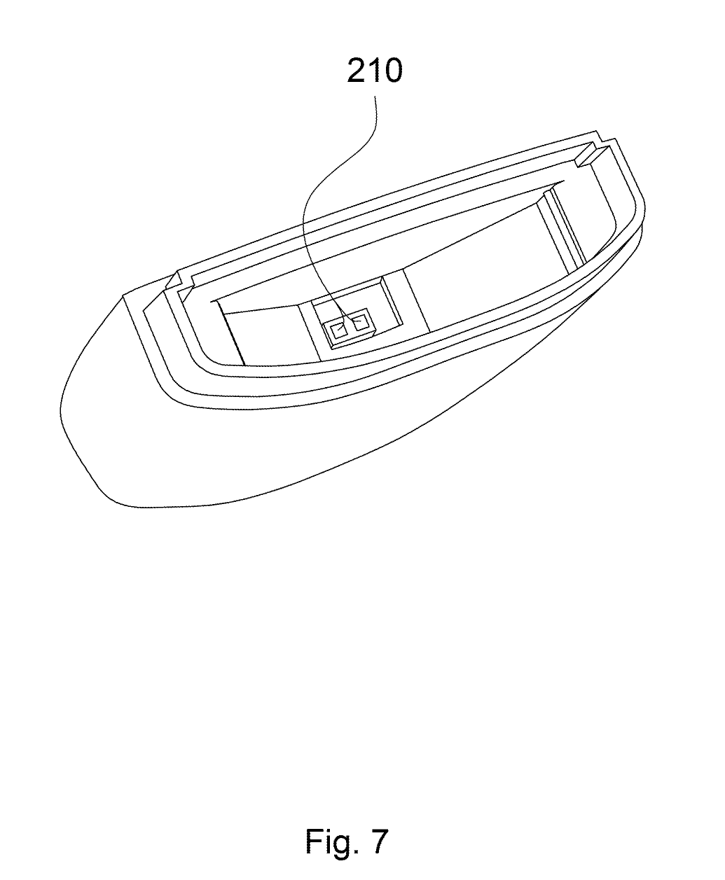

Refer to FIG. 6 and FIG. 7. The present invention also proposes an atomizer 1000, which further comprises a base seat 200 and the abovementioned atomizing assembly, which includes the containing part 110, the atomizing part 120 and the fixing cover 130. The base seat 200 includes a base electric-connection portion 210, which is fabricated into a structure able to be electrically connected with the atomizing electric-connection portion 121 of the atomizing part 120. The base electric-connection portion 210 can function as a power source or is electrically connected with a power source. Via the connection of the atomizing electric-connection portion 121 and the base electric-connection portion 210, the atomizing assembly 100 and the base seat 200 are assembled to form the atomizer 1000. In one embodiment, the atomizing electric-connection portion 121 is a group of pins; the atomizing assembly 100 is electrically connected with the base seat 200 in a pin-insertion way. According to requirement, the atomizer 1000 of the present invention can function as a sprayer of essence or perfume, a humidity modifier, a cosmetic moisturizer, or an applicator.

In conclusion, the present invention proposes an atomizing assembly, which comprises a containing part and an atomizing part respectively having a first pattern and a second pattern, wherein the first pattern is one-piece integrally-formed on the recess and the second pattern is one-piece integrally-formed on the nozzle plate. The first pattern and the second pattern are corresponding to each other and can be press-fitted to each other, whereby the containing part and the atomizing part can be detachably engaged with each other, and whereby the containing part and the atomizing part can be securely press-fitted to each other without using additional fixing element, wherefore the present invention can effectively avoid liquid leakage, and wherefore the present invention can increase the utilization efficiency of the liquid and decrease the malfunction rate of the device.

What is claimed is:

1. An atomizing assembly comprising:
a containing part including a containing body defining a containing space for containing a liquid to be atomized, wherein said containing body has a recess with a first pattern, the first pattern is one-piece integrally-formed on the recess; and
an atomizing part including:
a nozzle plate comprising a spray orifice portion disposed thereon;
an atomizing electric-connection portion electrically connected with said nozzle plate; and
an elastic element having two openings on the opposite side, said elastic element inseparable encapsulate said nozzle plate and expose said fine spray orifice portion by said two openings;
wherein said elastic element has a second pattern corresponding to said first pattern on one side, and said second pattern of said elastic element is closely press-fitted to said first pattern of said containing body to make said nozzle plate detachably installed in said recess of said containing body.

2. The atomizing assembly according to claim 1, wherein said first pattern is a convex pattern, and said second pattern is a concave pattern, and wherein said convex pattern and said concave pattern are corresponding to each other.

3. The atomizing assembly according to claim 1, wherein said first pattern is a concave pattern, and said second pattern is a convex pattern, and wherein said concave pattern and said convex pattern are corresponding to each other.

4. The atomizing assembly according to claim 2, wherein said first pattern has an annular shape, and said second pattern also has an annular shape.

5. The atomizing assembly according to claim 3, wherein said first pattern has an annular shape, and said second pattern also has an annular shape.

6. The atomizing assembly according to claim 1 further comprising a fixing cover, whose shape is corresponding to said nozzle plate and said atomizing electric-connection portion, and which is used to cover said nozzle plate and said atomizing electric-connection portion and fix said nozzle plate and said atomizing electric-connection portion to said containing part.

7. The atomizing assembly according to claim 1, wherein the said first pattern and said second pattern each include a plurality of concentric annular rings separated by annular gaps, and all of the annular rings of each pattern correspond in size to the annular gaps in the other pattern, the annular rings of largest radius being in the first pattern.

8. The atomizing assembly according to claim 1, wherein the said first pattern and said second pattern each include a plurality of concentric annular rings separated by annular gaps, and all of the annular rings of each pattern correspond in size to the annular gaps in the other pattern, the annular rings of largest radius being in the second pattern.

9. The atomizing assembly according to claim 1, wherein the material of said elastic element comprises rubber.

10. An atomizer comprising an atomizing assembly and a base seat, wherein the atomizing assembly comprising:
a containing part including a containing body defining a containing space for containing a liquid to be atomized, wherein said containing body has a recess with a first pattern, the first pattern is one-piece integrally-formed on the recess; and
an atomizing part including:
a nozzle plate comprising a spray orifice portion disposed thereon;
an atomizing electric-connection portion electrically connected with said nozzle plate; and
an elastic element having two openings, said elastic element inseparable encapsulate said nozzle plate and expose said fine spray orifice portion by said two openings;
wherein said elastic element has a second pattern on one side corresponding to said first pattern, and said second pattern of said elastic element is closely press-fitted to said first pattern of said containing body to make said nozzle plate detachably installed in said recess of said containing body;
wherein said base seat has a base electric-connection portion, and wherein said base electric-connection portion is fabricated into a structure able to be electrically connected with said atomizing electric-connection portion.

11. The atomizer according to claim 10, wherein said first pattern is a convex pattern, and said second pattern is a concave pattern, and wherein said convex pattern and said concave pattern are corresponding to each other.

12. The atomizer according to claim 10, wherein said first pattern is a concave pattern, and said second pattern is a convex pattern, and wherein said concave pattern and said convex pattern are corresponding to each other.

13. The atomizer according to claim 11, wherein said first pattern has an annular shape, and said second pattern also has an annular shape.

14. The atomizer according to claim 12, wherein said first pattern has an annular shape, and said second pattern also has an annular shape.

15. The atomizer according to claim 10, further comprising a fixing cover, whose shape is corresponding to said nozzle plate and said atomizing electric-connection portion, and which is used to cover said nozzle plate and said atomizing electric-connection portion and fix said nozzle plate and said atomizing electric-connection portion to said containing part.

16. The atomizer according to claim 10, wherein the said first pattern and said second pattern each include a plurality of concentric annular rings separated by annular gaps, and all of the annular rings of each pattern correspond in size to the annular gaps in the other pattern, the annular rings of largest radius being in the first pattern.

17. The atomizer according to claim 10, wherein the said first pattern and said second pattern each include a plurality of concentric annular rings separated by annular gaps, and all of the annular rings of each pattern correspond in size to the annular gaps in the other pattern, the annular rings of largest radius being in the second pattern.

18. The atomizer according to claim 10, wherein the material of said elastic element comprises rubber.
